(19) United States
(12) Patent Application Publication
(10) Pub. No.: US 2026/0248125 A1
(43) Pub. Date: Aug. 27, 2026

(54) AN ANTIBACTERIAL COMPOSITION AND METHOD OF FORMING THE SAME

(71) Applicants: SINGAPORE HEALTH SERVICES PTE LTD, Singapore (SG); SINGAPORE UNIVERSITY OF TECHNOLOGY AND DESIGN, Singapore (SG)

(72) Inventors: Ping WU, Singapore (SG); Franklin ANARIBA, Singapore (SG); A.J.M. Hasanthi Lakshika SENEVIRATHNA, Singapore (SG); Puwakdandawe Vishakha Thilini WEERASINGHE, Singapore (SG); Ashutosh AGARWAL, Singapore (SG); Wuen Pei Cathie LEE, Singapore (SG); Foo Cheong NG, Singapore (SG); Shie Lyeen Crystal WONG, Singapore (SG); Seok Hwee KOO, Singapore (SG); Joon Jae PARK, Singapore (SG)

(21) Appl. No.: 19/159,198

(22) PCT Filed: Feb. 20, 2024
(86) PCT No.: PCT/SG2024/050089

§ 371 Date: Aug. 22, 2025

(30) Foreign Application Priority Data
Feb. 24, 2023 (SG) 10202300495Q

(51) Int. Cl. A01N 25/12; A01N 59/00; A01P 1/00; B82Y 5/00

(52) U.S. Cl. CPC A01N 25/12; A01N 59/00; A01P 1/00; B82Y 5/00

(57) ABSTRACT

There is provided an antibacterial composition comprising: nanostructures of a Group (II) metal oxide-hydroxide composite. There is also provided a method of forming the antibacterial composition. In a particular embodiment, the Group (II) metal oxide-hydroxide composite may be magnesium oxide-hydroxide composite, calcium oxide-hydroxide composite, or a combination thereof.

### TECHNICAL FIELD

The present invention relates to an antibacterial composition and a method of forming thereof.

### BACKGROUND

Antibacterial surfaces are in high demand for biomedical applications to reduce infections associated with implanted medical devices. Conventional antibacterial compositions have caused concerns regarding their use, due to cytotoxic effects associated with the accumulation of heavy metal elements-based nanoparticles in the human body. Coating of biomedical devices with biocidal agents such as antibiotics is a conventional biochemical approach, however, excessive use of antibiotics renders bacteria drug-resistant and causes chronic infections.

Thus, there is a need for an improved antibacterial composition and method of forming the same.

### SUMMARY OF THE INVENTION

The present invention seeks to address these problems, and/or to provide an improved antibacterial composition and method of forming the same.

According to a first aspect, the present invention provides an antibacterial composition comprising: nanostructures of a Group (II) metal oxide-hydroxide composite.

According to a particular aspect, the composition may be applied to a support. The support may comprise a network of fibres. According to a particular aspect, the support may comprise reticulated foam.

According to a particular aspect, the nanostructures may be attached to the support. The nanostructures may comprise a plurality of arms, wherein adjacent arms have interstitial spaces extending therebetween. The plurality of arms may comprise at least one petaloid member.

According to a particular aspect, an average distance of the interstitial spaces between adjacent arms may be 90-200 nm. The plurality of arms may have an average thickness of 10-60 nm.

According to a particular aspect, the nanostructures may comprise nanoflowers.

According to a particular aspect, the Group (II) metal may be magnesium, calcium, or a combination thereof. In particular, the Group (II) metal may be magnesium.

The composition may have 2-hour bactericidal efficacy of >50% for Gram-positive and Gram-negative bacteria. The composition may have 24-hour bactericidal efficacy of >99% for Gram-positive and Gram-negative bacteria.

According to a second aspect, there is provided a method of forming the antibacterial composition, comprising:
mixing a Group (II) metal hydroxide solution and a polymeric solution to form a mixed solution;
forming nanofiber layers from the mixed solution;
calcining the nanofiber layers;
forming particles from the calcined nanofiber layers; and
steaming the particles to form the composition.

The forming nanofiber layers may comprise electrospinning. The steaming may be for 10-50 minutes.

According to a particular aspect, the method may comprise applying the composition to a support. The method may comprise pre-treating the support prior to the applying. The pre-treating may comprise contacting the support with an acid and a polysaccharide.

According to a particular aspect, the method may comprise drying the nanofiber layers prior to the calcining.

According to a particular aspect, the method may comprise drying the particles after the steaming.

### BRIEF DESCRIPTION OF THE DRAWINGS

In order that the invention may be fully understood and readily put into practical effect there shall now be described by way of non-limitative example only exemplary embodiments, the description being with reference to the accompanying illustrative drawings. In the drawings:

FIG. 1 shows XRD spectrum of MgO/Mg(OH)₂ composite;

FIG. 2 shows FTIR spectra of uncoated melamine sponge, MgO/Mg(OH)₂, and MgO/Mg(OH)₂ coated melamine sponge;

FIG. 3 shows SEM images; FIGS. 3A-3D show MgO/Mg(OH)₂ coated melamine sponge at different magnifications; FIGS. 3B″ and 3C″ show uncoated melamine sponge; FIG. 3E shows MgO/Mg(OH)₂ 3D nano flowers with distinct petals;

FIG. 4 shows histogram plots of distance between adjacent petals, and petals thickness;

FIG. 5 shows temporal variation in CFU count and Log(count) with and without MgO/Mg(OH)₂ 3D nano flowers coated melamine sponge; FIGS. 5A and 5A′ show CFU count and Log(count) of Staphylococcus aureus; FIGS. 5B and 5B′ show CFU count and Log(count) of Enterococcus faecalis; FIGS. 5C and 5C′ show CFU count and Log(count) of Escherichia coli; FIGS. 5D and 5D′ show CFU count and Log(count) of Klebsiella pneumonia; and FIGS. 5E and 5E′ show CFU count and Log(count) of Pseudomonas aeruginosa, error bars are 1 SD from the mean of triplicate measurements; and

FIG. 6 shows a schematic illustration of bactericidal mechanism of MgO/Mg(OH)₂ 3D nano flowers coated melamine sponge.

### DETAILED DESCRIPTION

As explained above, there is a need for an improved antibacterial composition and method of forming the same.

In general terms, the present invention provides an antibacterial composition comprising: nanostructures of a Group (II) metal oxide-hydroxide composite. The Group (II) metal oxide-hydroxide composite advantageously attracts a wider spectrum of bacteria types due to dual functionalities of hydrophilicity and hydrophobicity from the oxide and hydroxide groups. Hydrophilicity and hydrophobicity are key factors that determine the initial adhesion of bacteria cells on a surface. By understanding the multifaceted and complex interactions between bacterial cells and surfaces influenced by both the chemical and physical properties of the bacterial and surface materials, the present inventors have come up with an antibacterial composition and related surfaces that can beneficially prevent bacterial colonization for a wide spectrum of hygiene and health-related applications.

The antibacterial composition is also advantageously biocompatible and non-toxic, being able to be efficiently degraded and metabolized by human body, and thus eliminating any chance of excessive metal accumulation. This allows for further applications of the composition to medical devices.

According to a first aspect, the present invention provides an antibacterial composition comprising: nanostructures of a Group (II) metal oxide-hydroxide composite.

For the purposes of the present invention, the use of the singular includes the plural unless specifically stated otherwise. It should be noted that, as used in the specification and the appended claims, the singular forms “a”, “an” and “the” include plural referents unless the context clearly dictates otherwise. Further, the use of the term “including”, “comprising”, and “having” as well as other forms, such as “include”, “comprise”, “have” are not considered limiting.

For the purposes of the present invention, references to an antibacterial composition refers to a substance that kills bacteria and/or stops their growth.

The nanostructures may comprise a structure or an arrangement having at least one dimension on the nanoscale. The nanostructures may have one dimension on the nanoscale, two dimensions on the nanoscale, or three dimensions on the nanoscale. For the purposes of the present invention, references to nanoscale refers to a range of <1000 nm.

For the purposes of the present invention, references to a composite refers to a material which is produced from two or more constituent materials in which the individual elements remain separate and distinct within the finished structure. The composite may be a biphasic material comprising an oxide state and a hydroxide state of one or more Group (II) metals structure.

The composite may be a Group (II) metal oxide-hydroxide composite. For example, the composite may be a beryllium oxide-hydroxide composite, magnesium oxide-hydroxide composite, calcium oxide-hydroxide composite, strontium oxide-hydroxide composite, barium oxide-hydroxide composite, or any combination thereof. In particular, the composite may be magnesium oxide-hydroxide composite, calcium oxide-hydroxide composite, or a combination thereof.

According to a particular aspect, the Group (II) metal may be magnesium, calcium, or a combination thereof. Even more in particular, the Group (II) metal may be magnesium. Being biocompatible and non-toxic, as well as possessing superior mechanical properties, magnesium may advantageously be used in the antibacterial composition applied to bio-implants.

According to a particular aspect, the composition is applied to a support. The support may be any suitable support to enable attachment of the nanostructures to the support. For example, the support may have any suitable porosity and channels providing suitable permeability, with any suitable internal architecture which provides a large surface area for such attachment. In particular, the support may comprise a network of fibres. Even more in particular, the support may comprise reticulated foam. The reticulated foam advantageously provides a porous internal structure, while providing suitable mechanical support and ensuring uniform and homogenous attachment of nanostructures. The support may also be biocompatible and non-toxic. For example, the support may be an organic or inorganic polymer. In particular, the support may be a melamine sponge.

The support may have a porosity of >99%. The support may comprise pores with pore sizes ranging between 100-200 μm. In particular, the support may comprise pores with pore sizes ranging between 110-190 μm, 120-180 μm, 130-170 μm, 140-160 μm.

According to a particular aspect, the nanostructures may be attached to the support. The attachment may be any suitable attachment. For example, the attachment may be by electrostatic attachment, mechanical attachment, chemical attachment.

According to a particular aspect, the nanostructures may comprise a plurality of arms, wherein adjacent arms have interstitial spaces extending therebetween. The plurality of arms may have any suitable shape and dimensions. For example, the plurality of arms may be elongated. The plurality of arms may have sharp ends, rounded ends, tapered ends, straight ends. In particular, the plurality of arms may have sharp ends, with any suitable spacing between each of the ends. In particular, the plurality of arms may comprise at least one petaloid member.

The average distance of the interstitial spaces between adjacent arms may be any suitable distance to achieve bactericidal effect. For example, if the average distance is lower, the localized surface area of the arms in contact with the bacteria increases, which diminishes bacterial cell membrane stretching and limits bactericidal efficacy. On the other hand, if the average distance is higher, the membrane stretching is delocalized, which yet again reduces the bactericidal efficiency. The average distance of the interstitial spaces between adjacent arms may be 90-200 nm. In particular, the average distance may be 100-190 nm, 110-180 nm, 120-170 nm, 130-160 nm, 140-150 nm. Even more in particular, the average distance may be 100-140 nm, 110-130 nm.

The plurality of arms may have any suitable size in at least one dimension, and suitable rigidity, to achieve bactericidal effect. For example, the plurality of arms may have an average thickness smaller than a minimum dimension of bacteria, such that as the bacteria is pulled down towards the surface by adhesive and/or gravitational forces, the bacterial cell membrane is punctured by the nailing action provided by the rigidity of the plurality of arms. For example, the plurality of arms may have an elastic modulus of 1-10 GPa.

The plurality of arms may have an average thickness of 10-60 nm. In particular, the average thickness may be 15-55 nm, 20-50 nm, 25-45 nm, 30-40 nm. Even more in particular, the average thickness may be 25-45 nm.

The nanostructures may comprise nanoflowers, nanomeadows, nanotrees, nanopillars, nanotubes, nanocages, or a combination thereof. In particular, the nanostructures may comprise nanoflowers.

According to a particular aspect, the composition may have a 2-hour bactericidal efficacy of >50% for Gram-positive and Gram-negative bacteria. For example, the composition may have a 2-hour bactericidal efficacy of >55%, >60%, >65%, for Gram-positive and Gram-negative bacteria. According to a particular aspect, the composition may have a 24-hour bactericidal efficacy of >99% for Gram-positive and Gram-negative bacteria. For example, the composition may have a 24-hour bactericidal efficacy of >99.1%> 99.2%, >99.3%, >99.4%, >99.5%, >99.6%, >99.7%, >99.8%, >99.9%, for Gram-positive and Gram-negative bacteria. For the purposes of the present invention, references to bactericidal efficacy refers to the temporal reduction in colony forming units (CFU) over the stated time period. In particular, a 2-hour bactericidal efficacy of >50% refers to a reduction of 50% of CFU over 2 hours, a 24-hour bactericidal efficacy of >99% refers to a reduction of 50% of CFU over 24 hours, and so on.

The Gram-positive bacteria may be spherical-shaped. The Gram-positive bacteria may be Staphylococcus, Enterococcus, Macrococcus, Streptococcus, Micrococcus. In particular, the Gram-positive bacteria may be Staphylococcus aureus, Enterococcus faecalis.

The Gram-negative bacteria may be rod-shaped. The Gram-negative bacteria may be Escherichia, Klebsiella, Pseudomonas, Enterobacter, Salmonella, Shigella. In particular, the Gram-negative bacteria may be Escherichia coli, Klebsiella pneumonia, Pseudomonas aeruginosa.

The present composition is advantageously capable of attracting and killing both Gram-positive bacteria and Gram-negative bacteria, individually and simultaneously. Thus, the composition may be used in a wide variety of clinical applications to reduce risks associated with pathogenic bacterial infections in the medical field. At the same time, since a single composition is capable of targeting and eliminating both Gram-positive bacteria and Gram-negative bacteria, reliance on antibiotics can be greatly reduced, thereby preventing chronic infections from drug-resistant bacteria. As the present composition can attract and kill both Gram-positive bacteria and Gram-negative bacteria individually and simultaneously, this allows flexible adoption in various medical conditions and especially in secondary and/or co-infections.

According to a second aspect, the present invention provides a method of forming an antibacterial composition according to the first aspect, comprising:
mixing a Group (II) metal hydroxide solution and a polymeric solution to form a mixed solution;
forming nanofiber layers from the mixed solution;
calcining the nanofiber layers;
forming particles from the calcined nanofiber layers; and
steaming the particles to form the composition.

The Group (II) metal hydroxide solution may comprise a mixture of the Group (II) metal hydroxide and an acid. The acid may be acetic acid. The mixture of the Group (II) metal hydroxide and the acid may be subjected to ultrasonication prior to the mixing. The ultrasonication may be at a temperature of 50-60° C. In particular, the ultrasonication may be at a temperature of 52-58° C., 54-56° C. Even more in particular, the ultrasonication may be at a temperature of 55° C. The ultrasonication may be for 0.5-2 hours. In particular, the ultrasonication may be for 0.75-1.75 hours, 1-1.5 hours. Even more in particular, the ultrasonication may be for 1 hour.

The polymeric solution may comprise any suitable polymer. The polymer may comprise any suitable water-soluble polymer. The polymer may be a biocompatible and/or a non-toxic polymer. In particular, the polymer may be poly(vinyl alcohol).

The mixing may be in a ratio of 15:200 (v:v) of the Group (II) metal hydroxide solution to the polymeric solution. In particular, the volume ratio of the Group (II) metal hydroxide solution to the polymeric solution may be 15:150, 15:100. Even more in particular, the volume ratio of the Group (II) metal hydroxide solution to the polymeric solution in the mixed solution may be 15:100. The mixing may comprise ultrasonicating the mixed solution at any suitable temperature. For example, the ultrasonication may be at a temperature of 50-60° C. In particular, the ultrasonication may be at a temperature of 52-58° C., 54-56° C. Even more in particular, the ultrasonication may be at a temperature of 55° C. The ultrasonication may be for 10-30 minutes. In particular, the ultrasonication may be for 12-28 minutes, 14-26 minutes, 16-24 minutes. Even more in particular, the ultrasonication may be for 20 minutes.

The forming nanofiber layers may comprise any suitable forming method which produces the nanostructures comprised in the composite. For example, the forming may comprise electrospinning, dry spinning, wet spinning, gel spinning.

The calcining nanofiber layers may be at any suitable temperature. For example, the calcining may be at 300-500° C. In particular, the calcining may be at 325-475° C., 350-450° C., 375-425° C. Even more in particular, the calcining may be at 350-400° C. The calcining may be for any suitable time. For example, the calcining may be for 0.5-2 hours. In particular, the calcining may be for 0.75-1.75 hours, 1-1.5 hours. Even more in particular, the calcining may be for 1 hour. The heating rate may be any suitable heating rate. For example, the heating rate may be 2° C./min.

The forming particles may be by any suitable method to reduce the average size of the nanofiber layers. For example, the forming particles may be by grinding, crushing, cutting, ball milling. In particular, the forming particles may be by grinding with a mortar and pestle. The particles formed may be powder.

The steaming the particles may be by any suitable steaming method to expose the particles to steam. For example, the steaming may be by microwave steaming. The microwave steaming may be at 1300-2020 W. In particular, the microwave steaming may be at 1400-1900 W, 1500-1800 W, 1600-1700 W. Even more in particular, the microwave steaming may be at 1800 W. The steam exposure may lead to formation of Group (II) metal hydroxide, thus forming the Group (II) metal oxide-hydroxide composite.

According to a particular aspect, the forming nanofiber layers may comprise electrospinning. The polymer may facilitate electrospinning, acting as a medium for the Group (II) metal hydroxide solution to be electrospun. The electrospinning may be performed at any suitable solution flow rate and any suitable voltage. For example, the solution flow rate may be 0.2-0.4 mL/hour. In particular, the solution flow rate may be 0.3 mL/hour. The voltage may be 15-20 kV. In particular, the voltage may be 16-19 kV, 17-18 kV. Even more in particular, the voltage may be 17 kV.

The steaming may be for any suitable time. For example, the steaming may be for 10-50 minutes. In particular, the steaming may be for 15-45 minutes, 20-40 minutes, 25-35 minutes. Even more in particular, the steaming may be for 40 minutes.

According to a particular aspect, the method may further comprise applying the composition to a support. The applying may be any suitable application for attaching the nanostructures comprised in the composition to the support. The composition may be provided in aqueous suspension or solution form to facilitate the applying. The applying may comprise contacting the support with the composition. For example, the applying may comprise contacting the support with the composition for a first period of time. The applying may further comprise repeating the contacting for a subsequent period of time. In particular, the first period of time may be 10-30 minutes. Even more in particular, the first period of time may be 20 minutes. The second period of time may be 5-15 minutes. In particular, the second period of time may be 10 minutes.

According to a particular aspect, the method may further comprise pre-treating the support prior to the applying. The pre-treating may be any suitable pre-treating for modifying and/or activating the support so as to enable stronger attachment of the nanostructures comprised in the composition to the support. For example, the pre-treating may comprise contacting the support with an acid and a polysaccharide. The contacting the support with an acid and a polysaccharide may be sequential. For example, the pre-treating may comprise contacting the support with an acid for any suitable period of time, followed by contacting the support with a polysaccharide for any suitable period of time. The suitable period of time may each be 1-10 minutes, 2-9 minutes, 3-8 minutes, 4-7 minutes, 5-6 minutes. In particular, the suitable period of time may each be 5 minutes.

The acid may be any suitable acid. For example, the acid may be polyacrylic acid.

The polysaccharide may be any suitable polysaccharide. For example, the polysaccharide may be chitosan.

According to a particular aspect, the method may further comprise drying the nanofiber layers prior to the calcining. The drying may form a brittle layer which may facilitate the forming of flakes to provide a greater effective surface area for the calcining. The drying may be at any suitable temperature for any suitable period of time. For example, the drying may be at 60-110° C. In particular, the drying may be at 65-105° C., 60-100° C., 65-95° C., 70-90° C., 75-85° C. Even more in particular, the drying may be at 60° C. The drying may be for 12-36 hours. In particular, the drying may be for 14-34 hours, 16-32 hours, 18-30 hours, 20-28 hours, 22-26 hours. Even more in particular, the drying may be for 24 hours.

According to a particular aspect, the method may further comprise drying the particles after the steaming. The drying the particles may be at any suitable temperature. For example, the drying may be at room temperature. The drying the particles may be for any suitable period of time. For example, the drying may be for 12-36 hours. In particular, the drying may be for 14-34 hours, 16-32 hours, 18-30 hours, 20-28 hours, 22-26 hours. Even more in particular, the drying may be for 24 hours.

Having now generally described the invention, the same will be more readily understood through reference to the following example which is provided by way of illustration, and is not intended to be limiting.

### Example

### Materials and Methods

Magnesium hydroxide (reagent grade, 95%) and polyvinyl alcohol (PVA) (87-90% hydrolyzed, molecular weight 30,000-70,000), were purchased from Sigma-Aldrich while glacial acetic acid (analytical grade, 99.8%), polyacrylic acid (PAA) (25 wt. % in water), and chitosan were acquired from Scharlau, Alfa Aesar, and MP Biomedicals, respectively. Melamine sponge (MS) (brand: Vesta) was sourced from a local e-commerce company. All the chemicals were used as received without further purification.

### Synthesis of 3D Nanoflowers of MgO/Mg(OH)₂

0.25 g of Mg(OH)₂ was dissolved in 5 mL glacial acetic acid at 55° C. for 1 hour in an ultrasonication bath (brand: Elmasonic P, 37 KHz). An aqueous PVA solution (5% w/w) was prepared by dissolving PVA powder in deionized (DI) water at room temperature under continuous magnetic stirring for 12 hours at 600 rpm. Both solutions were mixed in a ratio of 15:100 (v:v) and ultrasonicated (37 KHz) at 55° C. for 20 minutes to obtain a clear solution.

This solution was then placed in a 5 mL Terumo® syringe equipped with a 21G×1/2″ gauge-size needle. Electrospinning was carried out with a needle-collector top-down configuration whereby the distance between the needle and the collector was maintained at 13 cm. The solution flow rate and applied DC voltage were set at 0.3 mL h⁻¹ and 17 kV, respectively.

The nanofiber layers were deposited on Al foil spread across the collector. The sample collected over Al foil was then dried in a hot air oven at 110° C. for 24 hours to obtain a brittle layer. The flakes collected after scratching this brittle layer were calcined in a muffle furnace (Nabertherm, Germany) at 350° C. for 1 hour. The heating rate of the furnace was set at 2° C. min⁻¹. After calcination, the samples were allowed to cool down naturally to room temperature and then ground to a fine powder using a mortar and pestle. Thereafter, the fine powder was subjected to steam for 40 minutes in a steam oven (Toshiba). After steaming, the powder was collected and dried at room temperature in a vacuum desiccator for 24 hours.

### Coating 3D Nanoflowers of MgO/Mg(OH)₂ Over Melamine Sponge

Commercial MS was cut into a cuboid (1 cm×1 cm×0.5 cm) using an art knife and soaked in freshly prepared 0.1 wt. % PAA aqueous solution (pH=1, adjusted with 1M HCl) for 5 minutes. The MS was thoroughly squeezed with a pair of tweezers and then soaked in 0.5 wt. % chitosan aqueous solution (pH=5, adjusted with 1M HCl) for another 5 minutes, followed by sequential squeezing multiple times during the entire process.

Through this process, PAA and chitosan bind together by electrostatic interaction and provide active sites for the deposition of nanoflowers. The MS was washed with DI water and immersed in 0.3 wt. % aqueous suspensions of MgO/Mg(OH)₂ 3D nanoflowers for 20 minutes. During this process, the MS was squeezed with a pair of tweezers and re-soaked in the aqueous suspension after 10 minutes. The 3D nanoflowers coated MS thus obtained was dried in a hot air oven at 60° C. for 6 hours.

### Characterization

### X-Ray Diffraction (XRD)

X-ray diffraction (XRD) diffractograms of MgO/Mg(OH)₂ were recorded with Bruker D8 Advance X-ray diffractometer equipped with LYNXEYE detector. The X-ray generator was operating at 40 kV and 25 mA. The diffractograms were recorded at angles (26) between 10° to 70°, with a step size of 0.03° (20) employing Cu Kα radiation (λ=1.5406 Å). The crystallite size (d) of MgO/Mg(OH)₂ nanoparticles was evaluated by Debye-Scherrer's formula (Eq. 1).

$$d = \frac{0.9 λ}{β cos θ} \qquad Eq . 1$$

where 0.9 represents the molecular shape factor, λ is the wavelength of Cu Kα radiation (1.5406 Å), β is the full width at half maxima (radians) and θ is the Bragg's diffraction angle (radians) corresponding to the most intense peak.

### Scanning Electron Microscopy (SEM)

Surface morphologies of the samples were examined by scanning electron microscope (SEM) (JEOL JSM-7600 F). The sample imaging was improved by sputter coating with Au. Sputter coating enriches the secondary electron signal required for topographic examination by inhibiting sample charging during SEM analysis. ImageJ software (version 1.53t) from the National Institute of Health, USA was used to analyze the SEM images. The elemental imaging of coated melamine sponge was carried out by energy-dispersive spectroscopy (EDS) (X-MaxN-50, Oxford Instruments) embedded within the SEM. Fourier transform infrared spectroscopy (FTIR) was conducted to determine the chemical structure of the samples. For this purpose, Agilent Cary 630 FTIR spectrometer was employed to collect FTIR spectra at a resolution of 1 cm⁻¹ between the wavelengths 650 to 4000 cm⁻¹ with 32 scans each.

Antibacterial tests Five different bacteria strains (S. aureus, E. faecalis, E. coli, K. pneumoniae, and P. aeruginosa) were used to ascertain the bactericidal effects of 3D nanoflowers coated MS. Each bacteria was sub-cultured onto trypticase soy agar (TSA) with 5% sheep blood plate and incubated for 18 to 24 hours before commencing the antibacterial study. A 0.5 McFarland bacterial suspension was prepared for each organism using a sterile saline solution.

Each MS weighing between 0.021 to 0.032 g was fully immersed in defined volumes between 1.68 to 2.56 mL (0.0125 g of MS per mL) of bacterial suspension with a concentration of 104 CFU/mL to 105 CFU/mL, and incubated for a total of 24 hours.

From each bacterial suspension, a sampling volume of 10 μL was collected at baseline (t=0) and defined intervals (t=2, 4, 8, and 24 h) of incubation, and plated onto culture plates in duplicates, which were then incubated for 18 to 24 hours before enumeration. All incubation was performed at 35° C. under aerobic conditions. The presence of any bacterial growth on the culture plates was enumerated and the average colony-forming unit (CFU) of the two plates was calculated for each bacteria.

A control suspension (bacteria only without the MS) was set up alongside each corresponding test suspension and sampled as per the time intervals above. Another control solution (sterile saline with MS) was also set up and sampled at t=0 and t=24 hours to control for sterility throughout the study.

### Results and Discussion

### X-Ray Diffraction (XRD)

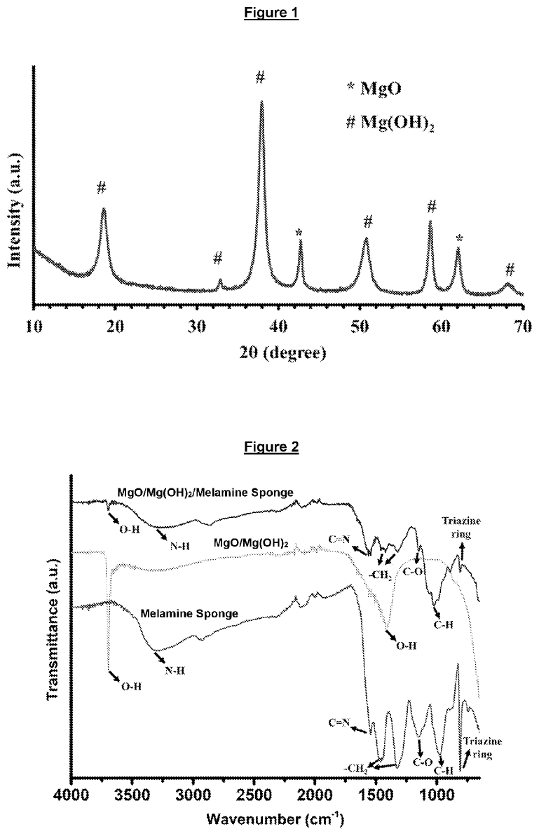

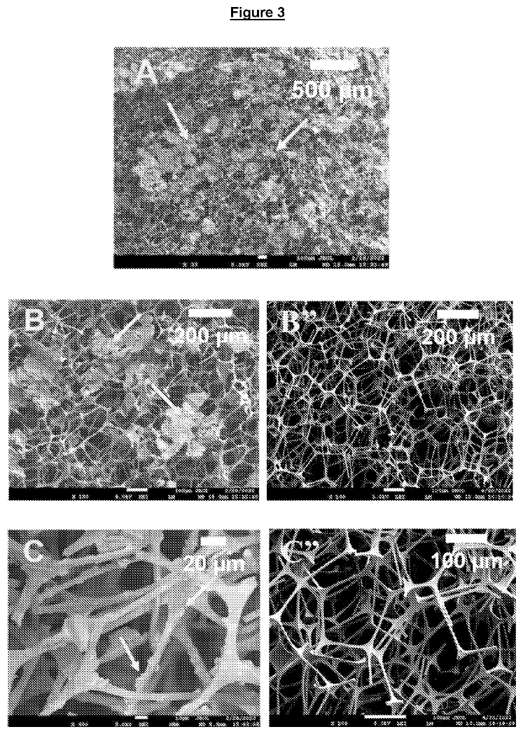

FIG. 1 shows powder XRD spectrum of MgO/Mg(OH)₂ prepared via electrospinning technique followed by calcination and steaming processes. The peaks at 20 values 18.5°, 33.0°, 37.9°, 50.7°, 58.6° and 68.1° correspond to (001), (100), (101), (102), (110) and (103) lattice planes of Mg(OH)₂ (ICDD 00-044-1482) while the two peaks at 20 values 42.9° and 62.3° refer to (200) and (220) lattice planes of MgO (ICDD 00-045-0946), respectively. The existence of diffraction peaks corresponding to both MgO and Mg(OH)₂ explicitly confirms the formation of composite MgO/Mg(OH)₂. The crystallite size as calculated from the most intense peak (37.9°) was found to be 8.8 nm. In addition, the high crystallinity of the synthesized nano material was apparent from the presence of intense XRD peaks.

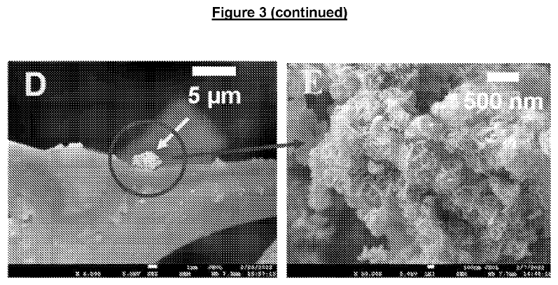

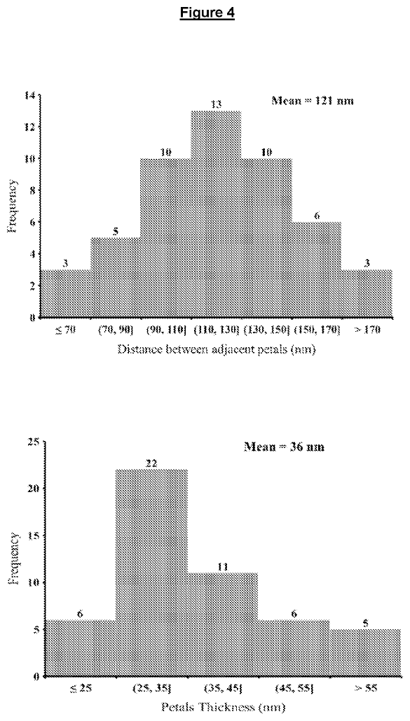

The Fourier-transform infrared spectroscopy (FTIR) spectra of MgO/Mg(OH)₂ 3D nano flowers, uncoated MS, and MgO/Mg(OH)₂ 3D nano flowers coated MS are shown in FIG. 2. The spectrum of composite MgO/Mg(OH)₂ displays sharp intense peak at 3697 cm⁻¹ and a distinct band at 1411 cm⁻¹. These were ascribed to the stretching (3697 cm⁻¹) and bending (1411 cm⁻¹) vibrations of the surface hydroxyl group. In the FTIR spectra of MgO/Mg(OH)₂ coated and uncoated MS, the peaks at 3312, 1559, 1460, 1313, 1145, 1013, 806 cm⁻¹ refer to the N—H stretching, C═N stretching, —CH₂— bending, C—O stretching, C—H bending and triazine ring bending vibrations, respectively. The presence of sharp O—H stretching peak at 3697 cm⁻¹ in the FTIR spectrum of MgO/Mg(OH)₂ 3D nano flowers coated MS confirms successful attachment of MgO/Mg(OH)₂ to the MS.

### Scanning Electron Microscopy (SEM)

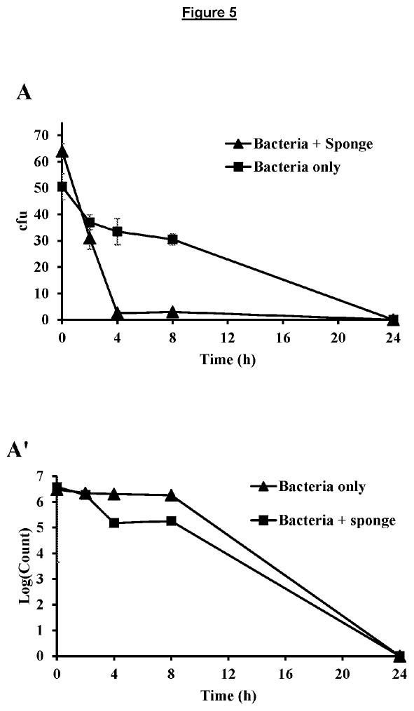

The morphologies of MgO/Mg(OH)₂ 3D nano flowers, uncoated MS and MgO/Mg(OH)₂ 3D nano flowers coated MS are shown in SEM images (FIG. 3). The uncoated MS exhibit highly inerratic 3D reticular structure with pore size ranging between 100 to 200 μm (FIGS. 3B″ and C″). The deposition of MgO/Mg(OH)₂ (marked by arrows) onto the MS skeleton is clearly visible in SEM images (FIGS. 3A, B, C and D). After coating with 3D nano flowers of MgO/Mg(OH)₂, the smooth MS surface became rough while the intrinsic 3D reticular structure of MS remained almost intact.

Subsequently, the color of MS changed from pure white to light brown. The magnified SEM image (FIG. 3E) of MgO/Mg(OH)₂ 3D nano flowers coated MS revealed the formation of distinct dense nano petals. Upon analysis with image analysis software (ImageJ), the mean petal thickness and mean distance between adjacent petals of 3D nano flowers were found to be 36 nm and 121 nm, respectively. Both these parameters were determined using a sample size of 50.

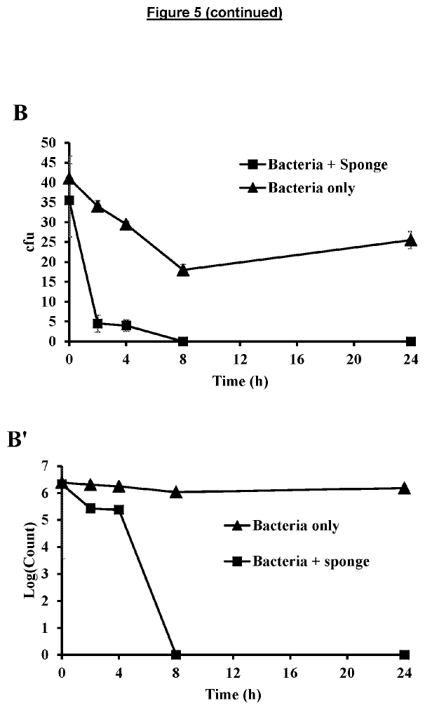

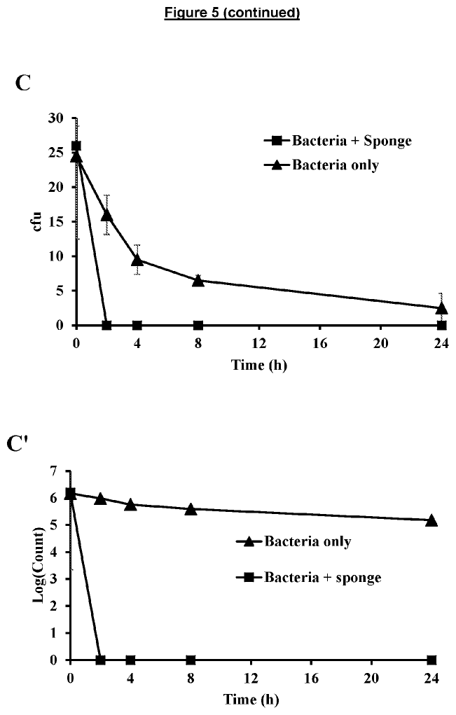

From the histogram plots (FIG. 4), it can be seen that the adjacent distance between majority of 3D nano flower petals range between 90 to 130 nm, while the petals' thicknesses vary from 25 to 45 nm. EDS mapping of the cross-sectional view of MgO/Mg(OH)₂ coated MS (not shown) revealed that the major elements Mg, C and O were uniformly distributed over the entire area. This demonstrates that the 3D nano flowers of MgO/Mg(OH)₂ were incorporated deep inside the inerratic 3D reticular structure of the MS. Indeed, the uniform light brown color of MgO/Mg(OH)₂ coated MS confirmed the homogeneous distribution of MgO/Mg(OH)₂ nano flowers throughout the 3D network of MS.

### Antibacterial Tests

The bactericidal efficacy of MgO/Mg(OH)₂ 3D nano flowers coated MS was determined over a period of 24 hours against five different bacteria, including two Gram-positive (S. aeureus and E. faecalis) and three Gram-negative (E. coli, K. pneumoniae and P. aeruginosa) bacteria.

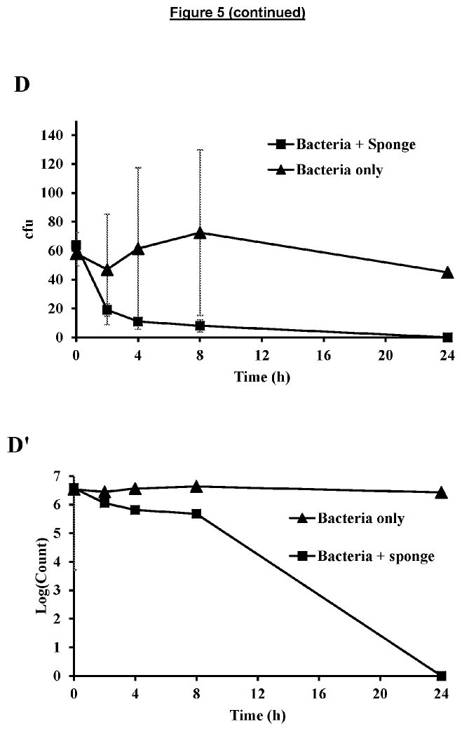

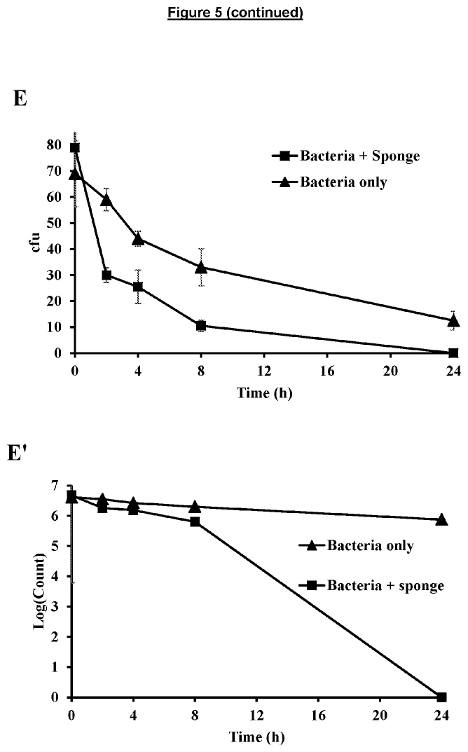

FIG. 5 shows the temporal variation in CFU and Log(count) of S. aeurus, E. faecalis, E. coli, K. pneumoniae and P. aeruginosa upon exposure to MgO/Mg(OH)₂ 3D nano flowers coated MS. The selected Gram-positive and Gram-negative bacteria were spherical-shaped and rod-shaped, respectively. The initial 2-hour bactericidal rate of 3D nano flowers coated MS follow the sequence: E. coli>P. aeruginosa>K. pneumoniae>S. aureus>E. faecalis.

From this trend, it is evident that in general the initial rate of bactericidal activity was higher for rod-shaped than spherical-shaped bacteria. This is attributed to large surface area of rod-shaped (Gram-negative) bacteria in direct contact with nano petals than spherical-shaped (Gram-positive) bacteria. In addition, thick and rigid cell walls of Gram-positive (spherical-shaped) bacteria allow them to withstand larger mechanical deformation compared to Gram-negative bacteria (rod-shaped), which usually have thin and fragile cell walls. Pertaining to this fact, the nano pillar structure of Psaltoda claripennis cicada wings also shows bactericidal activity only against Gram-negative bacterial strains.

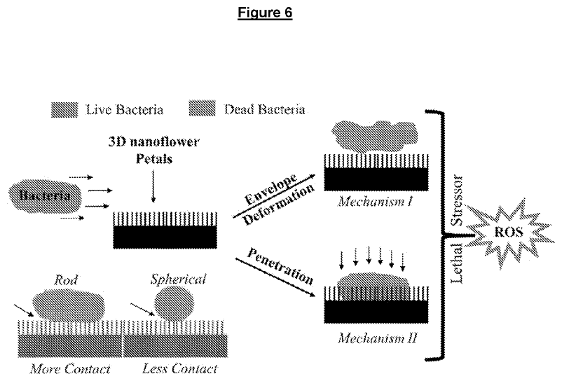

A plausible schematic illustration of the bactericidal mechanisms of 3D nano petals of MgO/Mg(OH)₂ coated melamine sponge is shown in FIG. 6. Since the mean distance (121 nm) between the adjacent petals of MgO/Mg(OH)₂ 3D nano flowers was much smaller than the minimum dimension (250 nm) of the bacteria, the bacteria could easily rest over the nano petals array without tumbling into the gorge between the adjacent petals. The increase in density of nano protrusions density increases the bacterial cell membrane permeability due to high stretching of the suspended envelope (Mechanism 1). Considering that the petals were substantially thinner (36 nm), it is expected that as the bacteria are pulled down towards the surface by adhesive and/or gravitational forces, the bacterial cell membrane is more likely to puncture by the nailing action of rigid nano petals (Mechanism II). The stiffness of nano protrusions in cicada wings have been known to play vital role in governing bacterial death by this mechanism. For instance, decrease in cicada wing nano pillar stiffness tend to bend the pillars when in contact with bacteria, in contrast to rigid pillars which favour bacterial deaths due to membrane rupture. Certainly, increased rigidity of nano protrusions favour bactericidal efficacy via puncture of cell membrane.

The bactericidal activity of MgO/Mg(OH)₂ 3D nano flower petals may also be ascribed to membrane stretching between nano petals which leads to deformation and eventual rupture of bacterial cell membrane. The spacing between nano protrusions governs the extent and severity of bactericidal activity. For example, if the nano petals are close enough, the localized surface area in contact with the bacteria also increases. This limits the bactericidal efficacy by diminishing bacterial cell membrane stretching. In contrast, if the nano petals are spaced substantially far apart, the membrane stretching is delocalized which yet again reduces the bactericidal efficiency. Hence, an optimal spacing between adjacent nano petals is highly desirable to achieve adequate bactericidal effect.

In the present example, the mean distance between the adjacent petals of MgO/Mg(OH)₂ 3D nano flowers was 121 nm. This was adequate to display substantial bactericidal activity. Apart from this, the movement of adhered bacteria across nano petals may also result in membrane buckling and shearing, leading to the separation of inner cytoplasmic cell membrane from the outer lipid bilayer.

Certainly, dense and sharp nano protrusions promote bacterial cell envelope deformation, penetration, and membrane permeability. Bacterial cells subjected to lethal stressors are induced by oxidative stress that triggers the formation of reactive oxygen species (ROS). Although several protective enzymes such as catalases, dismutase, etc. could detoxify low levels of ROS, if the ROS level crosses a certain threshold limit, bacterial death becomes inevitable and irreversible even after the initial stressor has been detached.

The 3D nano flowers of MgO/Mg(OH)₂ synthesized via electrospinning were successfully incorporated into commercial MS. After coating, the intrinsic 3D reticular structure of MS remained almost intact. The adjacent distance between majority of 3D nano flower petals ranged between 90 to 130 nm while petals' thicknesses varied from 25 to 45 nm. EDS mapping of the cross-sectional view of MgO/Mg(OH)₂ coated MS confirmed incorporation of 3D nano flowers of MgO/Mg(OH) deep inside the inerratic 3D reticular structure of MS. Thus, significant bactericidal activity of MgO/Mg(OH)₂ 3D nano flowers coated MS against both Gram-positive and Gram-negative bacteria has been demonstrated. It is expected that the novel bioinspired MgO/Mg(OH)₂ 3D nano flowers coated MS could be applied to reduce the risk associated with pathogenic bacterial infections in medical field.

Whilst the foregoing description has described exemplary embodiments, it will be understood by those skilled in the technology concerned that many variations may be made without departing from the present invention.

1. An antibacterial composition comprising: nanostructures of a Group (II) metal oxide-hydroxide composite.

2. The composition according to claim 1, wherein the composition is applied to a support.

3. The composition according to claim 2, wherein the support comprises a network of fibres.

4. The composition according to claim 2, wherein the support comprises reticulated foam.

5. The composition according to claim 2, wherein the nanostructures are attached to the support.

6. The composition according to claim 2, wherein the nanostructures comprise a plurality of arms, wherein adjacent arms have interstitial spaces extending therebetween.

7. The composition according to claim 6, wherein the plurality of arms comprise at least one petaloid member.

8. The composition according to claim 6, wherein an average distance of the interstitial spaces between adjacent arms is 90-200 nm.

9. The composition according to claim 6, wherein the plurality of arms have an average thickness of 10-60 nm.

10. The composition according to claim 1 wherein the nanostructures comprise nanoflowers.

11. (canceled)

12. (canceled)

13. The composition according to claim 1, wherein 2-hour bactericidal efficacy is >50% for Gram-positive and Gram-negative bacteria.

14. The composition according to claim 1, wherein 24-hour bactericidal efficacy is >99% for Gram-positive and Gram-negative bacteria.

15. A method of forming an antibacterial composition according to claim 1, comprising:
mixing a Group (II) metal hydroxide solution and a polymeric solution to form a mixed solution;
forming nanofiber layers from the mixed solution;
calcining the nanofiber layers;
forming particles from the calcined nanofiber layers; and
steaming the particles to form the composition.

16. The method according to claim 15, wherein the forming nanofiber layers comprises electrospinning.

17. The method according to claim 15, wherein the steaming is for 10-50 minutes.

18. The method according to claim 15, further comprising applying the composition to a support.

19. The method according to claim 18, further comprising pre-treating the support prior to the applying.

20. The method according to claim 19, wherein the pre-treating comprises contacting the support with an acid and a polysaccharide.

21. The method according to claim 15, further comprising drying the nanofiber layers prior to the calcining.

22. The method according to claim 15, further comprising drying the particles after the steaming.
